(19) United States
(12) Patent Application Publication
(10) Pub. No.: US 2006/0265780 A1
(43) Pub. Date: Nov. 23, 2006

(54) Interaction of glyphosate with photosystem II inhibitor herbicides as a selection tool for roundup ready events

(75) Inventors: S. Douglas Prosch, Ballwin, MO (US); Steven T. Voss, Edwardsville, IL (US)

Correspondence Address: SENNIGER POWERS (MTC), ONE METROPOLITAN SQUARE, 16TH FLOOR, ST LOUIS, MO, 63102, US

(73) Assignee: Monsanto Technology LLC, St. Louis, MO (US)

(21) Appl. No.: 11/418,772

(22) Filed: May 5, 2006

Related U.S. Application Data
(60) Provisional application No. 60/677,985, filed on May 5, 2005.

(51) Int. Cl. A01H 1/00; C12N 15/82

(52) U.S. Cl. 800/278

(57) ABSTRACT

A method of assessing herbicide tolerance in a plant is provided. The method of determining herbicide tolerance in plants comprises applying the herbicide to be tested in conjunction with at least one supplemental herbicide, determining the extent of resultant injury, and correlating the extent of injury to the herbicide tolerance of the plant.

### FIELD OF THE INVENTION

The present invention generally relates to assaying herbicide tolerance in plants. More particularly, the invention relates to assaying glyphosate tolerance in monocot or dicot plants, such as corn, rice, wheat, cotton, soybean, canola, peanut, bean, lentil, alfalfa and sunflower.

### BACKGROUND

Corn is an important crop and is a primary food source for humans and domesticated animals in many areas of the world. The methods of biotechnology have been applied to corn for improvement of the agronomic traits and the quality of the product. One such agronomic trait is herbicide tolerance, in particular, tolerance to glyphosate herbicide. This trait in corn can be conferred by the expression of a transgene in the corn plants.

The expression of foreign genes in plants is known to be influenced by their chromosomal position, perhaps due to chromatin structure (e.g., heterochromatin) or the proximity of transcriptional regulation elements (e.g., enhancers) close to the integration site. Weising et al., Ann. Rev. Genet (1988) 22, 421-477. For this reason, it is often necessary to screen a large number of events in order to identify an event characterized by optimal expression of an introduced gene of interest. For example, it has been observed in plants and in other organisms that there may be a wide variation in levels of expression of an introduced gene among events. There may also be differences in spatial or temporal patterns of expression, for example, differences in the relative expression of a transgene in various plant tissues, that may not correspond to the patterns expected from transcriptional regulatory elements present in the introduced gene construct.

For this reason, it is common to produce hundreds to thousands of different events and screen those events for a single event that has desired transgene expression levels and patterns for commercial purposes. An event that has desired levels or patterns of transgene expression is useful for introgressing the transgene into other genetic backgrounds by sexual outcrossing using conventional breeding methods. Progeny of such crosses maintain the transgene expression characteristics of the original transformant. This strategy is used to ensure reliable gene expression in a number of varieties that are well adapted to local growing conditions.

Herbicidal compositions comprising the herbicide N-phosphonomethyl-glycine, or derivatives thereof (“glyphosate”), are useful for suppressing the growth of, or killing, unwanted plants such as grasses, weeds, and the like. Glyphosate inhibits the shikimic acid pathway which leads to the biosynthesis of aromatic compounds including amino acids and vitamins. Specifically, glyphosate inhibits the conversion of phosphoenolpyruvic acid and 3-phosphoshikimic acid to 5-enolpyruvyl-3-phosphoshikimic acid by inhibiting the enzyme 5-enolpyruvyl-3-phosphoshikimic acid synthase (EPSP synthase or EPSPS). This leads to depletion of key amino acids that are necessary for protein synthesis and plant growth. Glyphosate typically is applied to and is absorbed by the foliage of the target plant. Glyphosate translocates upward in xylem and downward in phloem, generally causing injury to new growth. Plant foliage treated with glyphosate will first yellow (new leaves first) and then turn brown and die within 10-14 days after herbicide application.

Resistance to glyphosate can be obtained in a plant by introducing a transgene encoding EPSPS, especially when the transgene encodes a glyphosate insensitive EPSPS enzyme. Thus, as the herbicide glyphosate functions to kill the cell by interrupting aromatic amino acid biosynthesis, particularly in the cell's chloroplast, the expression of the EPSPS sequence fused to a chloroplast transit peptide sequence allows increased resistance to the herbicide by concentrating what glyphosate resistance enzyme the cell expresses in the chloroplast, i.e. in the target organelle of the cell. Exemplary herbicide resistance enzymes include EPSPS and glyphosate oxido-reductase (GOX) genes (see Comai, 1985, U.S. Pat. No. 4,535,060, specifically incorporated herein by reference in its entirety).

Chlorosis in newly expanding leaves of Roundup Ready plants can occur following an application of glyphosate. This is referred to as “yellow flash” because it is typically expressed in a transitory fashion. This phenomena is especially pronounced in soybean leaves, where chlorosis may occur in the newest expanding trifoliate and sometimes the subsequent trifoliate, but then normally disappears as the plant continues to grow. These symptoms are most often seen in the field under high growth conditions. For soybean, this situation can be easily duplicated in the greenhouse and consistent expression of “yellow flash” is obtained following application of glyphosate.

“Yellow flash” occurs much less frequently and, historically, has been more difficult to reproduce in Roundup Ready corn. Numerous greenhouse studies with various Roundup Ready corn hybrids have failed to show this symptomology on a consistent basis or, when it does occur, at a high level of expression.

Current selection of Roundup Ready corn events requires field testing in order to discern relative differences in glyphosate tolerance. This is due to the fact that early vegetative tolerance of these events to glyphosate is very high and crop injury is often not seen until the V8 stage of growth or later. While most plant tolerance to herbicides is generally directly related to plant size (i.e., large plants are harder to kill than small ones), corn tolerance to many herbicides is known to decrease with increasing plant size. This may be tied to a rapid change in corn leaf cuticle properties from the V5 to V8 stage (see Hennig-Gizewski and Wirth, Pflanzenschutz Nachrichten Bayer (2000) 53, 105-125) (noting that corn was the only plant species studied that had these rapid changes in cuticle characteristics, and the response was consistent with several hybrids and with plants grown in the field or in greenhouses). The “V” stage describes the number of lowermost leaves with visible collars; for example, at V4, there are four leaves with visible collars. Ear shoot initiation and tassel formation in corn are usually completed around the V5 stage. These reproductive structures are often sensitive to herbicides.

The selection of new Roundup Ready corn events based upon tolerance to glyphosate has been difficult due to the fact that greenhouse/growth chamber assays have not been effective at discerning various levels of tolerance. Typically, new Roundup Ready corn events require testing in the field where differential tolerance is only observed at the 8 leaf growth stage or later. For example, the NK 603 Roundup Ready corn hybrid at the 4-leaf and 6-leaf stage is known to be highly tolerant of high rates of glyphosate, high rates of glyphosate with ammonium sulfate, high rates of glyphosate applied to corn under cold stress, and sequential applications of high glyphosate rates to corn, with no injury symptomology, such as chlorosis or necrosis. As such, it has previously been difficult to use early injury expression as a selection tool for glyphosate resistance in corn.

### SUMMARY OF THE INVENTION

Among the various aspects of the present invention is an assay that can allow for discrimination of herbicide tolerance in different transgenic plant events at an earlier stage, and preferably in greenhouse/growth chamber testing, with substantial cost and time savings. The process of the present invention is particularly advantageous in connection with discrimination of glyphosate tolerance. This assay can act as a selection tool to discriminate among various Roundup Ready events based upon consistent injury symptomology.

Briefly, therefore, the present invention is directed to a process for assaying herbicide tolerance in a plant. The process comprises applying a herbicide for which tolerance is being tested in conjunction with at least one supplemental herbicide to a plant, determining the extent of resultant injury, and correlating the extent of injury to the tested-herbicide tolerance of the plant.

In one embodiment, the tested herbicide is glyphosate and the at least one supplemental herbicide is photosystem II (PSII) inhibitor.

In another embodiment, the plant being tested is a monocot. In still another embodiment, the plant being tested is a dicot.

Other objects and features will be in part apparent and in part pointed out hereinafter.

### BRIEF DESCRIPTION OF THE DRAWINGS

FIG. 1 is a bar graph showing percent growth inhibition 10 days after treatment of 2 corn event hybrids (DK 580 hybrid with the GA 21 event and DKC-53-33 hybrid with the NK 603 event) as a function of the type (Lorox or Sencor) and concentration (56, 112, or 224 gm/ha) of Photosystem II inhibitor in conjunction with application of 840 gm/ha of glyphosate. Methodology is described in Example 1.

FIG. 2 is a bar graph showing percent growth inhibition 10 days after treatment of 2 corn event hybrids (DK 580 hybrid with the GA 21 event and DKC-53-33 hybrid with the NK 603 event) as a function of the type (Lorox or Sencor) and concentration (56, 112, or 224 gm/ha) of Photosystem II inhibitor in conjunction with application of 1680 gm/ha of glyphosate. Methodology is described in Example 1.

FIG. 3 is a bar graph showing percent growth inhibition 10 days after treatment of 2 corn event hybrids (DK 580 hybrid with the GA 21 event and DKC-53-33 hybrid with the NK 603 event) as a function of the type (Lorox or Sencor) and concentration (56, 112, or 224 gm/ha) of Photosystem II inhibitor in conjunction with application of 3360 gm/ha of glyphosate. Methodology is described in Example 1.

FIG. 4 is a bar graph showing percent growth inhibition 11 days after treatment of 2 corn event hybrids (RX686 Roundup Ready hybrid with the GA 21 event and DKC-53-33 hybrid with the NK 603 event) as a function of the type (Lorox or Sencor) and concentration (56, 112, or 224 gm/ha) of Photosystem II inhibitor in conjunction with application of 1680 gm/ha of glyphosate. Methodology is described in Example 2.

FIG. 5 is a bar graph showing percent growth inhibition 11 days after treatment of 2 corn event hybrids (RX686Roundup Ready hybrid with the GA 21 event and DKC-53-33 hybrid with the NK 603 event) as a function of the type (Lorox or Sencor) and concentration (56, 112, or 224 gm/ha) of Photosystem II inhibitor in conjunction with application of 2520 gm/ha of glyphosate. Methodology is described in Example 2.

FIG. 6 is a bar graph showing percent growth inhibition 11 days after treatment of 2 corn event hybrids (RX686Roundup Ready hybrid with the GA 21 event and DKC-53-33 hybrid with the NK 603 event) as a function of the type (Lorox or Sencor) and concentration (56, 112, or 224 gm/ha) of Photosystem II inhibitor in conjunction with application of 3360 gm/ha of glyphosate. Methodology is described in Example 2.

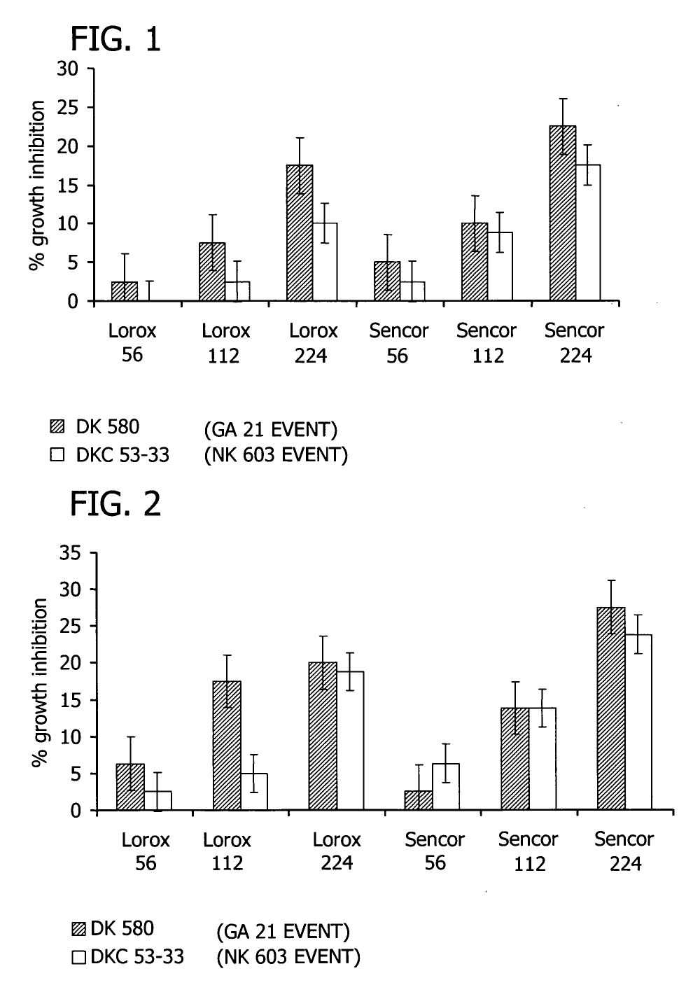

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

“Glyphosate” refers to N-phosphonomethylglycine and its salts. Glyphosate is the active ingredient of Roundup® herbicide (Monsanto Co, St. Louis, Mo.). Treatments with “glyphosate herbicide” refer to treatments with Roundup®, Roundup Ultra®, or Roundup UltraMAX® herbicides or any other formulation containing glyphosate. For the purposes of the present invention, the term “glyphosate” includes any herbicidally active form of N-phosphonomethylglycine (including any salt thereof) and other forms that result in the production of the glyphosate anion in plants. Treatments with “glyphosate” refer to treatments with the Roundup or Roundup Ultra herbicide formulation, unless otherwise stated. Additional formulations with herbicide activity that contain N-phosphonomethylglycine or any of its salts are herein included as a glyphosate herbicide.

Herbicide tolerance refers to the ability of a fraction of transformed plants, i.e., plants with at least one selectable event to survive a concentration of the herbicide which kills essentially all untransformed plants of the same species under the same conditions.

As used herein, a Roundup Ready event confers a substantial degree of glyphosate resistance (i.e., glyphosate tolerance) upon a plant if it allows a selectable fraction of transformed plants to survive a concentration of glyphosate which kills essentially all untransformed plants under the same conditions.

An “event” is the insertion of a particular transgene into a specific location on a chromosome. The three factors that differentiate events are: (i) the identity of the inserted transgene; (ii) the locus of insertion; and (iii) the copy number inserted at that locus. A transgenic corn event is produced by transformation of a corn plant cell with heterologous DNA, i.e., a nucleic acid construct that includes a transgene of interest, perpetuation of the event from cell to cell when the chromosome replicates and the cells divide, regeneration of a population of plants resulting from the insertion of the transgene into the genome of the plant, and selection of a particular plant characterized by insertion into a particular genome location. An event in the context of a transgenic corn event refers to DNA from the original transformant and progeny thereof comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA. Even after repeated back-crossing to a recurrent parent, the inserted DNA and flanking DNA from the transformed parent is present in the progeny of the cross at the same chromosomal location.

### DETAILED DESCRIPTION OF THE INVENTION

The inventors have observed that application of glyphosate in conjunction with a photosystem II (PSII) inhibitor results in a degree of injury that correlates with glyphosate tolerance in corn plants. While such glyphosate/PSII inhibitor assays can generally be used for determining the glyphosate tolerance of a variety of agriculturally important species, they offer particular advantage in determining the glyphosate tolerance of corn events, in which there tends to be greater difficulty in assessing young plants.

The present invention provides a method of assaying herbicide tolerance in a plant by growing the plant until a predetermined developmental age or for a predetermined interval of time, applying a herbicide for which tolerance is being tested to the plant, applying at least one supplemental herbicide for which tolerance is not being tested to the plant, determining extent of injury to the plant, and correlating the extent of injury to the plant's tolerance for the tested herbicide. In several embodiments of the invention, no significant injury is observed with application of the tested herbicide or the supplemental herbicide alone; however, the application of these herbicides together demonstrates an interaction prompting injury in the plant. In other embodiments, the application of the tested herbicide or the supplemental herbicide results in some measurable amount of injury when applied independently, and further, the application of these herbicides together increases the measurable injury of the plant. The differential injury response is correlated to the plant's tolerance to the tested herbicide.

In addition to testing plant tolerance to glyphosate, the present method allows for determining plant tolerance to other herbicides as well. Once a herbicide for which tolerance is being tested is selected, one skilled in the art can select a supplemental herbicide based on its mode of action. The supplemental herbicide is selected in such a manner as to enhance the effect of the tested herbicide so that a plant treated with these herbicides exhibits pronounced injury, which can be correlated to the plant's tolerance to the tested herbicide. A number of different combinations of a tested herbicide and a supplemental herbicide for use in the method of the present invention are shown in Table 1.

TABLE 1

| Tested Herbicide | Supplemental Herbicide |
|---|---|
| Glyphosate | PSII inhibitor |
| Glyphosate | ALS inhibitor |
| Glyphosate | Carotenoid biosynthesis inhibitor |
| Glyphosate | 4-HPPD inhibitor |
| Glyphosate | PPO inhibitor |
| PSII inhibitor | Glyphosate |
| PSII inhibitor | Synthetic auxin |
| PSII inhibitor | PPO inhibitor |
| PSII inhibitor | Photosystem I inhibitor |
| PSII inhibitor | ALS inhibitor |
| PSII inhibitor | Carotenoid biosynthesis inhibitor |
| ALS inhibitor | Glutamine synthesis inhibitor |
| ALS inhibitor | Glyphosate |
| ALS inhibitor | Synthetic auxin |
| ALS inhibitor | PSII inhibitor |
| ALS inhibitor | Acetamide |
| Synthetic auxin | Carotenoid biosynthesis inhibitor |
| Synthetic auxin | Diterpene inhibitor |
| Synthetic auxin | ALS inhibitor |
| Synthetic auxin | PSII Inhibitor |
| ACCase inhibitor | Thiocarbamate |
| ACCase inhibitor | Ethofumesate |
| ACCase inhibitor | Dalapon |
| Microtubule inhibitor | Acetamide |
| Microtubule inhibitor | Thiocarbamate |
| Microtubule inhibitor | ACCase inhibitor |
| Acetamide | Dinitroaniline |
| Acetamide | Microtubule inhibitor |
| Acetamide | Lipid synthesis inhibitor |
| Acetamide | Thiocarbamate |
| Acetamide | ALS inhibitor |
| Lipid synthesis inhibitor | ACCase inhibitor |
| Lipid synthesis inhibitor | Acetamide |
| Lipid synthesis inhibitor | Dinitroaniline |
| PPO inhibitor | PSII inhibitor |
| PPO inhibitor | Glyphosate |
| PPO inhibitor | Carotenoid biosynthesis inhibitor |
| Photosystem I inhibitor | Carotenoid biosynthesis inhibitor |
| Photosystem I inhibitor | PSII inhibitor |
| Photosystem I inhibitor | 4-HPPD inhibitor |
| Glutamine synthesis inhibitor | PSII inhibitor |
| Glutamine synthesis inhibitor | 4-HPPD inhibitor |
| Glutamine synthesis inhibitor | ALS inhibitor |
| Glutamine synthesis inhibitor | Glyphosate |
| Carotenoid biosynthesis inhibitor | Synthetic auxin |
| Carotenoid biosynthesis inhibitor | PPO inhibitor |
| Carotenoid biosynthesis inhibitor | Photosystem I inhibitor |
| Carotenoid biosynthesis inhibitor | Diterpene Inhibitor |
| Carotenoid biosynthesis inhibitor | 4-HPPD inhibitor |
| Diterpene inhibitor | Carotenoid biosynthesis inhibitor |
| Diterpene inhibitor | Synthetic auxin |
| Diterpene inhibitor | 4-HPPD inhibitor |
| 4-HPPD inhibitor | Carotenoid biosynthesis inhibitor |
| 4-HPPD inhibitor | Glutamine synthesis inhibitor |
| 4-HPPD inhibitor | Photosystem I inhibitor |
| 4-HPPD inhibitor | Diterpene inhibitor |
| 4-HPPD inhibitor | Glyphosate |
| Thiocarbamate | ACCase inhibitor |
| Thiocarbamate | Microtubule inhibitor |
| Thiocarbamate | Acetamide |
| Dinitroaniline | Acetamide |
| Dinitroaniline | Lipid synthesis inhibitor |
| ACCase inhibitor | Lipid synthesis inhibitor |
| ACCase inhibitor | Microtubule inhibitor |
| Ethofumesate | ACCase inhibitor |
| Dalapon | ACCase inhibitor |

In several of the above embodiments, the tested herbicide is glyphosate. As mentioned previously, glyphosate may be, for example, N-phosphonomethylglycine, a salt or adduct thereof, or a compound which is converted to glyphosate in plant tissues or which otherwise provides glyphosate ion. In this regard it is to be noted that the term “glyphosate,” when used herein, is to be understood to encompass such derivatives unless the context requires otherwise.

Glyphosate salts that can be used according to this invention include but are not restricted to, for example, alkali metal salts (e.g., sodium and potassium salts), ammonium salts, alkylammonium salts (e.g., C1-16 alkylammonium), alkanolammonium salts (e.g., C1-16 alkanolammonium), di-ammonium salts (e.g., dimethylammonium), alkylamine salts (e.g., dimethylamine and isopropylamine salts), alkanolamine salts (e.g., ethanolamine salts), alkylsulfonium salts (e.g., C1-16 alkylsulfonium, for example trimethylsulfonium salts), sulfoxonium salts, and mixtures or combinations thereof. For some embodiments, preferred glyphosate salts include for example the potassium salt, isopropylamine salt, ammonium salt, di-ammonium salt, sodium salt, monoethanolamine salt, and trimethylsulfonium salt.

Suitable commercially available glyphosate includes glyphosate (Sequence, Touchdown 009, Touchdown Total), diammonium glyphosate (Touchdown, Touchdown CF, Touchdown Pro), isopropylamine glyphosate (Accord, Accord XRT, AquaMaster, Backdraft SL, Campaign, Credit Duo, Credit Duo Extra, Credit Master, Credit Systemic, Credit Systemic Extra, Durango, Expert, Extra Credit 5, Extreme, Field Master, Forza, Glyfos, Glyfoa Aquatic, Glyfos X-tra, Glyfos Pro, GlyKamba Broad Spectrum, Glyphomax, Glyphomax Plus, Glyphomax XRT, Glypro, Glypro Plus, Honcho, Honcho Plus, Imitator Plus, Journey, Landmaster BW, Landmaster II, OneStep, Polado L, Ranger PRO, Rattler, Rattler Plus, RazorBurn, Recoil, Riverdale Aqua Neat, Riverdale Foresters, Riverdale Razor, Riverdale Razor Pro, Rodeo, RoundUp Original, RoundUp Original II, RoundUp Pro, RoundUp UltraMAX, RoundUp UltraMAX RT, RT Master), monoammonium glyphosate (Credit Duo, Credit Duo Extra, QuikPRO, RoundUp Pro Dry, RoundUp Ultra Dry), and potassium glyphosate (RoundUp Original MAX, RoundUp UltraMAX II, RoundUp WeatherMAX, RT Master II, Touchdown CT, Touchdown HiTech).

The herbicidal properties of N-phosphonomethylglycine and its derivatives were first discovered by Franz, then disclosed and patented in U.S. Pat. No. 3,799,758. A number of herbicidal salts of N-phosphonomethylglycine were patented by Franz in U.S. Pat. No. 4,405,531. The disclosures of both of these patents are hereby incorporated by reference.

Glyphosate compositions useful to the invention may be formulated with one or more surfactants to enhance their effectiveness for foliar application. When water is added to a composition formulated with surfactants, the resulting sprayable composition more easily and effectively covers the foliage (e.g., the leaves or other photosynthesizing organs) of plants. Glyphosate salts, for example, have been formulated with surfactants such as polyoxyalkylene-type surfactants including, among other surfactants, polyoxyalkylene alkylamines. Commercial formulations of glyphosate herbicide marketed under the trademark Roundup® have been formulated by Monsanto with such a polyoxyalkylene alkylamine, in particular a polyoxyethylene tallowamine.

In several of the above embodiments, the supplemental herbicide is a photosystem II (PSII) inhibitor. Generally, PSII inhibitors block electron transport and the transfer of light energy through binding to the D1 quinone protein of photosynthetic electron transport. PSII inhibitor herbicides cause injury through photooxidative and photoradical reactions in chloroplasts resulting in membrane rupture.

Examples of useful classes of PSII inhibitors include substituted ureas, triazines, uracils, phenyl-carbamates, pyridazinones, benzothiadiazoles (bentazon), nitriles (bromoxynil), and phenyl-pyridazines (pyridate).

Examples of triazines include metribuzin (Sencor4, Sencor 75DF, Lexone, Axiom, Axiom AT, Axiom DF, Boundary, Canopy, Domain, Metribuzin 4, Metribuzin 75DF, Turbo), atrazine (Aatrex, Atra-5, Atrazine 4L, Atrazine 90DF, Atrazine 90WSP, Axiom AT, Basis Gold, Banvel K+ atrazine, Bicep group, Buctril+atrazine, Bullet, Cinch, Contour, Cy-Pro AT, Degree Xtra, Double Team, Expert, Extrazine II, Field Master, FulTime, Guardsman, Harness Xtra, Keystone, Laddok S-12, Lariat, Lexar, LeadOff, Liberty ATZ, Lumax, Marksman, Parallel Plus, Pro-mate atrazine, Simazat 4L, Ready Master ATZ, Stalwart Xtra, Steadfast ATZ, Shotgun, Surpass 100, Trizmet II), cyanazine (Bladex, Cy-Pro, Cy-Pro AT, Extrazine II), hexazinone (Velpar, Velpar AlfaMax MP, Oustar, Westar), prometryne (Caparol, Gesagard, Cotton pro, Suprend), ametryn (Evik), and simazine (Simazat, Simazine 90DF, Simadex, Princep, Princep Caliber, Princep Liquid, SIM-TROL 4L, SIM-TROL 90DF). A preferable triazine is metribuzin. Triazines translocation occurs only upwards in the xylem. Photosynthesis inhibitors do not usually prevent seedlings from germinating or emerging. Injury symptoms of triazines occur after the cotyledons and first true leaves emerge. Injury symptoms include chlorosis and necrosis at leaf tips and margins on older leaves first (lower leaves) followed by interveinal chlorosis and lower leaf drop. Older and larger leaves will be affected first because they take up more of the herbicide from the water solution and they are the primary photosynthetic tissue of the plant. Injured leaf tissue will eventually turn necrotic. Because of the chemical nature of the herbicide-soil relationship, injury symptoms are likely to increase as soil pH increases (above 7.2).

Examples of substituted ureas include linuron (Afolan, Lorox, Layby pro, Linex 4L), diuron (Dibro 4+4, Direx, Diuron 4L, Diuron 80DF, Ginstar EC, Krovar I DF, Riverdale Dibro 2+2, Riverdale Dibro 4+2, Karmex, Sahara DG, Thidiazuron-Diuron EC, Velpar Alfamax MP), metobromuron (Patoran), fluometuron (Cotoran, Lanex), tebuthiuron (Graslan, Spike), and monolinuron (Afesin). A preferable substituted urea is linuron. Substituted ureas and uracils are xylem mobile, bind to D1 quinone protein of photosynthetic electron transport, and have similar symptoms as for triazines.

Examples of phenyl-carbamates include desmedipham (Betamix, Betamix beta, Betanex, Betanex beta, Progress, Progress beta) and phenmedipham (Spin-Aid, Betamix, Betamix beta, Betanex, Betanex beta, Progress, Progress beta). An example of a pyridazinone is pyrazon (Pyramin). Examples of uracils include bromacil (Hyvar, Krovar, Riverdale Dibro 2+2, Riverdale Dibro 4+2, Dibro 4+4) and terbacil (Sinbar). An example of a benzothiadiazole is bentazon (Basagran, Conclude Xact, Laddok S-12, Rezult B). An example of a nitrile is bromoxynil (Bromox MCPA 2-2, Bronate, Bronate Advanced, Brominal, Buctril, Buctril 4 Cereals, Buctril 4EC, Buctril+atrazine, Connect 20 WSP, Double Up B+D, Maestro D, Maestro MA, Starane NXTcp, Pardner, Wildcat Xtra). An example of a phenyl-pyridazine is pyridate (Lentagran, Tough).

With some PSII inhibitors, such as bentazon, bromoxynil, and pyridate (contact), injury is confined to foliage that has come in contact with the herbicide (i.e., on leaves that are emerged at the time of treatment but not on new leaves emerging after treatment). Affected leaves will become yellow or bronze in color, occasionally have brown mid-veins, and will eventually turn necrotic. Low doses of these herbicides mimic classical photosynthesis inhibitors. High doses mimic cell membrane disrupters. Crop oil concentrates, other additives, and warm weather may intensify crop injury symptoms. Grass plants are generally tolerant to the non-systemic photosynthesis inhibitors.

Suitable inhibitors of acetyl CoA carboxylase (ACCase) include aryloxyphenoxys (clodinafop), propionates (cyhalofop-butyl, diclofop, fenoxaprop, fluazifop-P, haloxyfop, propaquizafop, or quizalofop-P), and cyclohexanediones (alloxydim, butroxydim, clethodim, cycloxydim, sethoxydim, or tralkoxydim).

Inhibitors of acetolactate synthase (ALS) which are suitable include imidazolinones (imazamethabenz, imazamox, imazapic, imazapyr, imazaquin, or imazethapyr), pyrimidinylthio-benzoates (bispyribac-sodium, pyrithiobac, or pyribenzoxim), sulfonylzminocarbonyl-triazolinones (flucarbazone-sodium, or propoxycarbazone), sulfonylureas (amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron-methyl, foramsulfuron, halosulfuron, iodosulfuron, metsulfuron, nicosulfuron, primisulfuron, prosulfuron, pyrazosulfuron-ethyl, rimsulfuron, sulfometuron, sulfosulfuron, thifensulfuron, triasulfuron, tribenuron, trifloxysulfuron sodium, or triflusulfuron), and triazolopyrimidines (cloransulam-methyl, diclosulam, florasulam, or flumetsulam).

Microtubule assembly inhibitors useful in the methods of the invention include dinitroanilines (benefin, ethalfluralin, oryzalin, pendimethalin, prod iamine, or trifluralin), pyridines (dithiopyr or thiazopyr), and DCPA.

Suitable synthetic auxins include phenoxys (2,4-D, 2,4-DB, dichlorpropr, 2,4-DP, MCPA, MCPB, or mecoprop, PP), benzoic acids (dicamba), carboxylic acids (clopyralid, fluroxypyr, picloram, or triclopyr), and quinaline carboxcylic acids (quinclorac).

Thiocarbamates which are suitable include butylate, cycloate, EPTC, esprocarb, molinate, pebulate, prosulfocarb, thiobencarb, triallate, and vernolate.

Inhibitors of carotenoid biosynthesis for use in the inventive methods include triazoles (amitrole or aclonifen) as well as beflubtiamid, fluridone, flurochloridone, flurtamone, pyridazinones (norflurazon), and pyrininecarboxamides (diflufenican or picolinafen).

Suitable inhibitors of protoporphyrinogen oxidase (PPO) include diphenylethers (acifluorfen, bifenox, fomesafen, fluroglycofen, lactofen, or oxyfluorfen), N-phenylphthalimides (fluthiacet, flumiclorac, or flumioxazin), as well as flufenpyr-ethyl, oxadiazoles (oxadiazon, oxadiargyl, or sulfentrazone), phenylpyrazoles (pyrafllufen-ethyl), pyrimidindiones (butafenacil), thiadiazoles (fluthiacet-methyl), and triazolinones (azafenidin, or carfentrazone-ethyl).

Acetamides that are suitable include napropamide, chloroacetamides (acetochlor, alachlor, butachlor, dimethenamid, metolachlor, metazachlor, pretilachlor, propachlor, or thenylchlor), and oxyacetamides (mefenacet or flufenacet).

Suitable photosystem I inhibitors include bipyridyliums such as diquat or paraquat.

Inhibitors of 4-hyrroxyhenyl-pyruvate-dioxygenase (4-HPPD) for use in the methods of the invention include callistemones (mesotrione), isoxazoles (isoxaflutole), pyrazoles (benzofenap, pyrazolynate, or pyrazoxyfen), and triketones (sulcotrione).

It has been demonstrated that in some plants, bean or pea for example, treatment with sub-lethal doses of glyphosate effect pronounced interveinal chlorosis in the youngest leaves. Researchers have suggested that the glyphosate induced chlorosis is linked to detrimental effects on the synthesis of aminolevulinic acid (ALA), a precursor in the synthesis of chlorophyll. See Grossbard and Atkinson (1985) The Herbicide Glyphosate, Butterworth & Co., p. 36. Glyphosate strongly inhibits synthesis of chlorophyll and its precursor aminolevulinic acid (ALA) via inhibition of the incorporation of glutamate, 2-ketoglutarate, and glycine into ALA. Kitchen, Witt, and Reick (1981) Weed Sci., 29, 513-516.

In contrast, PSII inhibitors block electron transport, hindering the reduction of plastoquinone, and as a result, absorbed excitation energy cannot be disposed of in the normal fashion. In this situation, chlorophyll accumulates in the more stable triplet state. The accessory pigment β-carotene can quench some of the excited triplet chlorophyll and re-emit the absorbed energy in a nonradiative manner. See generally Siefermann-Harms, Physiol. Plant. (1987) 69, 561-568. While this and other quenching pathways are efficient and adequate under normal conditions, the energy quenching ability is overloaded in herbicidally inhibited leaves, allowing the excess triplet chlorophyll to react with oxygen to form reactive oxygen species. These reactive oxygen species can induce pigment bleaching and lipid peroxidation. Know and Dodge Phytochemistry (1985) 24, 889-896. Initial visual injury is manifested as chlorosis and, with a sub-lethal dose of a photosystem II inhibitor, this may be the only symptomology evident.

While not being bound by any particular mechanism, it is thought that the combination of a PSII inhibitor and glyphosate in a glyphosate tolerant crop such as corn would impact chlorophyll from two different directions. One would lead to chlorophyll damage and the other would inhibit chlorophyll synthesis. While neither herbicide alone would necessarily cause visual injury symptoms, the combination of the two would be capable of inducing sufficient injury to produce rate-dependent chlorotic symptomology.

It will be evident to a skilled artisan that a large number of different glyphosate and PSII inhibitor combinations can be made. By way of example, metribuzin can be used as a supplemental herbicide in combination with glyphosate. Similarly, linuron can be used to supplement glyphosate in the present method. Table 2 lists a number of different glyphosate/PSII inhibitor combinations that can be used in the present method.

TABLE 2

| Tested Herbicide | Supplemental Herbicide |
|---|---|
| Potassium glyphosate | Ametryne |
| Potassium glyphosate | Atrazine |
| Potassium glyphosate | Cyanazine |
| Potassium glyphosate | Simazine |
| Potassium glyphosate | Hexazinone |
| Potassium glyphosate | Metribuzin |
| Potassium glyphosate | Terbacil |
| Potassium glyphosate | Diuron |
| Potassium glyphosate | Linuron |
| Potassium glyphosate | Tebuthiuron |
| Potassium glyphosate | Bromoxynil |
| Potassium glyphosate | Bentazon |
| Potassium glyphosate | Pyridate |
| Monoammonium glyphosate | Ametryne |
| Monoammonium glyphosate | Atrazine |
| Monoammonium glyphosate | Cyanazine |
| Monoammonium glyphosate | Simazine |
| Monoammonium glyphosate | Hexazinone |
| Monoammonium glyphosate | Metribuzin |
| Monoammonium glyphosate | Terbacil |
| Monoammonium glyphosate | Diuron |
| Monoammonium glyphosate | Linuron |
| Monoammonium glyphosate | Tebuthiuron |
| Monoammonium glyphosate | Bromoxynil |
| Monoammonium glyphosate | Bentazon |
| Monoammonium glyphosate | Pyridate |
| Diammonium glyphosate | Ametryne |
| Diammonium glyphosate | Atrazine |
| Diammonium glyphosate | Cyanazine |
| Diammonium glyphosate | Simazine |
| Diammonium glyphosate | Hexazinone |
| Diammonium glyphosate | Metribuzin |
| Diammonium glyphosate | Terbacil |
| Diammonium glyphosate | Diuron |
| Diammonium glyphosate | Linuron |
| Diammonium glyphosate | Tebuthiuron |
| Diammonium glyphosate | Bromoxynil |
| Diammonium glyphosate | Bentazon |
| Diammonium glyphosate | Pyridate |
| Sodium glyphosate | Ametryne |
| Sodium glyphosate | Atrazine |
| Sodium glyphosate | Cyanazine |
| Sodium glyphosate | Simazine |
| Sodium glyphosate | Hexazinone |
| Sodium glyphosate | Metribuzin |
| Sodium glyphosate | Terbacil |
| Sodium glyphosate | Diuron |
| Sodium glyphosate | Linuron |
| Sodium glyphosate | Tebuthiuron |
| Sodium glyphosate | Bromoxynil |
| Sodium glyphosate | Bentazon |
| Sodium glyphosate | Pyridate |
| Monoethanolamine glyphosate | Ametryne |
| Monoethanolamine glyphosate | Atrazine |
| Monoethanolamine glyphosate | Cyanazine |
| Monoethanolamine glyphosate | Simazine |
| Monoethanolamine glyphosate | Hexazinone |
| Monoethanolamine glyphosate | Metribuzin |
| Monoethanolamine glyphosate | Terbacil |
| Monoethanolamine glyphosate | Diuron |
| Monoethanolamine glyphosate | Linuron |
| Monoethanolamine glyphosate | Tebuthiuron |
| Monoethanolamine glyphosate | Bromoxynil |
| Monoethanolamine glyphosate | Bentazon |
| Monoethanolamine glyphosate | Pyridate |
| N-propylamine glyphosate | Ametryne |
| N-propylamine glyphosate | Atrazine |
| N-propylamine glyphosate | Cyanazine |
| N-propylamine glyphosate | Simazine |
| N-propylamine glyphosate | Hexazinone |
| N-propylamine glyphosate | Metribuzin |
| N-propylamine glyphosate | Terbacil |
| N-propylamine glyphosate | Diuron |
| N-propylamine glyphosate | Linuron |
| N-propylamine glyphosate | Tebuthiuron |
| N-propylamine glyphosate | Bromoxynil |
| N-propylamine glyphosate | Bentazon |
| N-propylamine glyphosate | Pyridate |
| Isopropylamine glyphosate | Ametryne |
| Isopropylamine glyphosate | Atrazine |
| Isopropylamine glyphosate | Cyanazine |
| Isopropylamine glyphosate | Simazine |
| Isopropylamine glyphosate | Hexazinone |
| Isopropylamine glyphosate | Metribuzin |
| Isopropylamine glyphosate | Terbacil |
| Isopropylamine glyphosate | Diuron |
| Isopropylamine glyphosate | Linuron |
| Isopropylamine glyphosate | Tebuthiuron |
| Isopropylamine glyphosate | Bromoxynil |
| Isopropylamine glyphosate | Bentazon |
| Isopropylamine glyphosate | Pyridate |
| Ethylamine glyphosate | Ametryne |
| Ethylamine glyphosate | Atrazine |
| Ethylamine glyphosate | Cyanazine |
| Ethylamine glyphosate | Simazine |
| Ethylamine glyphosate | Hexazinone |
| Ethylamine glyphosate | Metribuzin |
| Ethylamine glyphosate | Terbacil |
| Ethylamine glyphosate | Diuron |
| Ethylamine glyphosate | Linuron |
| Ethylamine glyphosate | Tebuthiuron |
| Ethylamine glyphosate | Bromoxynil |
| Ethylamine glyphosate | Bentazon |
| Ethylamine glyphosate | Pyridate |
| Ethylenediamine glyphosate | Ametryne |
| Ethylenediamine glyphosate | Atrazine |
| Ethylenediamine glyphosate | Cyanazine |
| Ethylenediamine glyphosate | Simazine |
| Ethylenediamine glyphosate | Hexazinone |
| Ethylenediamine glyphosate | Metribuzin |
| Ethylenediamine glyphosate | Terbacil |
| Ethylenediamine glyphosate | Diuron |
| Ethylenediamine glyphosate | Linuron |
| Ethylenediamine glyphosate | Tebuthiuron |
| Ethylenediamine glyphosate | Bromoxynil |
| Ethylenediamine glyphosate | Bentazon |
| Ethylenediamine glyphosate | Pyridate |
| Hexamethylenediamine glyphosate | Ametryne |
| Hexamethylenediamine glyphosate | Atrazine |
| Hexamethylenediamine glyphosate | Cyanazine |
| Hexamethylenediamine glyphosate | Simazine |
| Hexamethylenediamine glyphosate | Hexazinone |
| Hexamethylenediamine glyphosate | Metribuzin |
| Hexamethylenediamine glyphosate | Terbacil |
| Hexamethylenediamine glyphosate | Diuron |
| Hexamethylenediamine glyphosate | Linuron |
| Hexamethylenediamine glyphosate | Tebuthiuron |
| Hexamethylenediamine glyphosate | Bromoxynil |
| Hexamethylenediamine glyphosate | Bentazon |
| Hexamethylenediamine glyphosate | Pyridate |
| Trimethylsulfonium glyphosate | Ametryne |
| Trimethylsulfonium glyphosate | Atrazine |
| Trimethylsulfonium glyphosate | Cyanazine |
| Trimethylsulfonium glyphosate | Simazine |
| Trimethylsulfonium glyphosate | Hexazinone |
| Trimethylsulfonium glyphosate | Metribuzin |
| Trimethylsulfonium glyphosate | Terbacil |
| Trimethylsulfonium glyphosate | Diuron |
| Trimethylsulfonium glyphosate | Linuron |
| Trimethylsulfonium glyphosate | Tebuthiuron |
| Trimethylsulfonium glyphosate | Bromoxynil |
| Trimethylsulfonium glyphosate | Bentazon |
| Trimethylsulfonium glyphosate | Pyridate |

Two or more PSII inhibitors can be used to supplement glyphosate in the present method. By way of example, linuron and metribuzin, metribuzin and atrazine, linuron and diuron, diuron, atrazine and cyanazine are some of the exemplary combinations of PSII inhibitors that can be used.

Herbicide compositions useful in the invention can be prepared simply by diluting a concentrate herbicide composition in water. The herbicidal spray compositions included in the present invention can be applied to the foliage of the plants to be treated through any of the appropriate methods that are well known to those having skill in the art. Application of herbicide treatment solutions to foliage can be accomplished, for example, by spraying with any conventional means for spraying liquids, such as spray nozzles, atomizers, or the like.

Furthermore, the combinations according to the invention may be employed together with other active compounds, for example from the group of safeners, fungicides, insecticides, and plant growth regulators, or from the group of the additives and formulation auxiliaries which are customary in crop protection.

A tested herbicide and a supplemental herbicide, such as glyphosate and the PSII inhibitor, are applied to a plant jointly or sequentially. An example of joint application is application via a tank mix. In another embodiment, the two herbicides are applied at different times (e.g., splitting). In a further embodiment, the herbicides, such as glyphosate and the PSII inhibitor are applied in a plurality of portions (e.g., sequential application).

In one embodiment, both herbicides are applied at a concentration not sufficient to injure the plant if each was applied alone. In another embodiment, the tested and the supplemental herbicides are applied at a concentration sufficient to injure the plant if each was applied alone. In a further embodiment, the tested herbicide is applied at a concentration not sufficient to injure the plant if applied alone, while the supplemental herbicide is applied at a concentration sufficient to injure the plant if applied alone. In still another embodiment, the tested herbicide is applied at a concentration sufficient to injure the plant if applied alone, while the supplemental herbicide is applied at a concentration not sufficient to injure the plant if applied alone; Preferably, the tested herbicide is glyphosate and the supplemental herbicide is a PSII inhibitor.

In several embodiments of the invention, no significant injury is observed with application of glyphosate or the PSII inhibitor alone; however, the application of these compounds together demonstrates an interaction prompting injury in a plant, such as Roundup Ready corn. In other embodiments, the application of glyphosate or PSII inhibitor results in some measurable amount of injury when applied independently, and further, the application of these compounds together increases the measurable injury of the plant. The differential injury response is then correlated to the plant's tolerance to glyphosate. In a preferred embodiment, the level of plant injury is inversely correlated with glyphosate tolerance.

The present method of assaying tolerance to a herbicide is applicable to a number of different plants, such as monocots and dicots. In one embodiment, the monocot plants are selected from corn, rice, wheat, barley, oat, rye, buckwheat, sugar cane, onion, banana, date, and pineapple. Preferably, the monocot plant is selected from corn, rice and wheat. In another preferred embodiment, the monocot plant is corn. Alternatively, the dicot plants are selected from the group consisting of cotton, soybeans, canola, beans, lentils, peanuts, sunflower, broccoli, alfalfa, clover, carrots, strawberries, raspberries, oranges, apples, cherries, plums, parsley, coriander, dill, and fennel. Preferably, the dicots are selected from cotton, soybeans, beans, lentils, peanuts, alfalfa and sunflower. More preferably, the dicot plants are selected from cotton and soybeans.

While any plant may be assayed according to the methods described herein, these methods are especially useful for assaying plants with potential glyphosate tolerance due to the insertion of a glyphosate tolerance event into the plant's genome or the genome of its progenitors. Accordingly, in some of the embodiments, the plant comprises Roundup Ready events or is a progeny thereof. By way of example, the Roundup Ready plant is selected from Roundup Ready corn, Roundup Ready soybeans, Roundup Ready cotton, Roundup Ready wheat and Roundup Ready alfalfa. Preferably, the plant is a Roundup Ready corn. The generation, selection, and genotypic/phenotypic testing of such Roundup Ready corn events is further described in, for example, the commonly assigned U.S. Pat. No. 5,554,798 entitled “Fertile glyphosate-resistant transgenic corn plants,” the disclosure of which is specifically incorporated herein by reference.

According to various embodiments of the present invention, a plant which is being tested for tolerance for a particular herbicide is planted and grown in a greenhouse, growth chamber, or field and treated with a sufficient amount of a tested herbicide and a supplemental herbicide to result in measurable damage. In one embodiment, corn seed comprising Roundup Ready events, or progeny thereof are planted and treated as described. In another embodiment, the tested herbicide is glyphosate and the supplemental herbicide is a PSII inhibitor. The measurable damage resulting from the application of herbicides is then correlated to the tolerance of the plant event for the tested herbicide.

According to various embodiments of the assay, after planting of the seed of interest, the resulting plant is grown for a predetermined time or until a predetermined age before the application of a tested herbicide and a supplemental herbicide. In one embodiment, the plant is a corn plant, the tested herbicide is glyphosate and the supplemental herbicide is a PSII inhibitor. After supplemental herbicide application, the treated plant is allowed to grow for an additional predetermined time or until a second predetermined age. Various combinations of developmental age and chronological time are useful to the invention.

To some extent, the choice of how long to allow the plant to grow will depend upon growing conditions, the particular hybrid being assayed, the tested herbicide formulation used, the supplemental herbicide used, the injury symptom being assessed, and other factors as commonly understood by those skilled in the art. In one embodiment, when correlating growth inhibition to glyphosate tolerance, a corn plant can be grown until about growth stage 11 (about one leaf unfolded) or about growth stage 12 (about two leaves unfolded) before application of glyphosate and PSII inhibitor(s), and then grown for about 2 to about 15 days after application. For example, a corn plant can be grown about 5 to about 10 days after the application of glyphosate and PSII inhibitor. As another example, a corn plant can be grown about 8 days after the application of glyphosate and PSII inhibitor. Similar time periods can be used for growing soybeans, cotton, canola, and other crops. In addition, one of ordinary skill in the art can readily determine the suitable time periods for which particular plants should be grown.

As is known in the art, a variety of plant development indices are useful to assess plant developmental age. Examples of such developmental indices useful for monocots include, but are not limited to the Leaf Collar Method, the “Droopy” leaf method, and the Extended BBCH scale. In corn, the Leaf Collar Method determines leaf stage by counting the number of leaves on a plant with visible leaf collars, beginning with the lowermost, short, rounded-tip true leaf and ending with the uppermost leaf with a visible leaf collar. The leaf collar is the light-colored collar-like “band” located at the base of an exposed leaf blade, near the spot where the leaf blade comes in contact with the stem of the plant. Leaves within the whorl, not yet fully expanded and with no visible leaf collar are generally not included in this leaf staging method. Leaf stages are usually described as “V” stages, e.g., V2=two leaves with visible leaf collars. The leaf collar method is a widely used agronomy method, especially in the U.S. See generally Ritchie et al. 1992, How a corn plant develops, Sp. Rpt. #48, Iowa State University of Science and Technology, Cooperative Extension Service, Ames, Iowa.

The Extended BBCH scale is a system for uniform coding of phenologically identical stages of monocotyledonous plant species. The decimal code, which is divided into principal and secondary growth stages (GS), is based on the well-known cereal code developed by Zadoks et al. (1974), a decimal code for the growth stages of cereals. Weed Res. 14:415-421. Principal growth stage 0 (00-09) describes the stages of germination. Principal growth stage 1 (10-19) describes leaf development. For example, at GS 11, there is one leaf unfolded, while at GS 12, there are two leaves unfolded. Principal growth stages 2-9 describe tillering, stem elongation, booting, heading, flowering, fruiting, ripening, and senescence, respectively. The Extended BBCH scale is further described in Stauss 1994, Compendium of Growth Stage Identification Keys for Mono- and Dicotyledenous Plants, Ciba-Geigy AG, ISBN 3-9520749-0-X. Application.

Various embodiments of the invention are generally directed at screening plants for glyphosate resistance. Because many of the screened plants have at least some glyphosate resistance, often glyphosate applied alone at herbicidally effective amounts will be insufficient to significantly harm the assayed plant. But according to the methods of the invention, application of glyphosate in conjunction with a PSII inhibitor can effect herbicide injury symptoms in the assayed plant. This expression of injury can then be correlated to glyphosate tolerance of the assayed plant.

In several embodiments, glyphosate is applied at a herbicidally effective rate. Generally, a herbicidally effective rate is sufficient to effect visual symptoms of glyphosate treatment in non-glyphosate tolerant plants within two to seven days after treatment. Depending upon the glyphosate tolerance of the assayed plant, a herbicidally effective rate of glyphosate applied without a PSII inhibitor may or may not effect visual symptoms in the assayed plant.

The selection of application rates that are herbicidally effective for a tested herbicide or supplemental herbicide of the invention is within the skill of the ordinary agricultural scientist. Those of skill in the art will likewise recognize that individual plant conditions, weather and growing conditions, as well as the specific active ingredients and their weight ratio in the composition, will influence the degree of herbicidal effectiveness achieved in practicing this invention. With respect to the use of glyphosate compositions, much information is known about appropriate application rates. Over two decades of glyphosate use and published studies relating to such use have provided abundant information from which a practitioner can select glyphosate application rates that are herbicidally effective on particular species at particular growth stages in particular environmental conditions.

In several embodiments, glyphosate can be applied from about 1× to about 4× of suggested field rates. These application rates are usually expressed as amount of glyphosate per unit area treated, e.g. grams per hectare (gm/ha). In one embodiment, glyphosate is applied at a concentration of about 840 gm/ha to about 3360 gm/ha. For example, glyphosate can be applied at a concentration of about 840 gm/ha. As another example, glyphosate can be applied at a concentration of about 1680 gm/ha. As a further example, glyphosate can be applied at a concentration of about 2520 gm/ha. As yet another example, glyphosate can be applied at a concentration of about 3360 gm/ha.

According to various embodiments of the invention, a PSII inhibitor is applied in conjunction with glyphosate, resulting in measurable damage which can then be correlated to glyphosate resistance. In several embodiments, the PSII inhibitor is applied at a concentration not sufficient to significantly injure the plant when applied independently. As an example, a PSII inhibitor can be applied at ¼× field rate for corn. As another example, a PSII inhibitor can be applied at ½× field rate. In one embodiment, the PSII inhibitor is applied at a concentration of about 56 gm/ha to about 224 gm/ha. As an example, the PSII inhibitor can be applied at a concentration of about 56 gm/ha. As another example, the PSII inhibitor can be applied at a concentration of about 112 gm/ha. As a further example, the PSII inhibitor can be applied at a concentration of about 224 gm/ha.

With respect to herbicide combinations other than glyphosate and PSII inhibitor, the tested and the supplemental herbicides can be applied at the rates similar to those of glyphosate and PSII inhibitor. For example, when the tested herbicide is an ALS inhibitor, it can be applied at a field rate from about 1× to about 4×, whereas the supplemental herbicide (e.g., glyphosate) can be applied at a field rate from about ¼× to about ½×. Suitable field rates for particular combinations of the tested herbicide and the supplemental herbicide can be readily determined by one of ordinary skill in the art.

According to various embodiments of the invention, several physiological or developmental stress symptoms resultant from the tested herbicide (e.g., glyphosate) and the supplemental herbicide (e.g., a PSII inhibitor) application can be measured, and this value correlated to the tested-herbicide tolerance (e.g., glyphosate tolerance) of the plant. So, for example, after growing the plant to be assayed, applying the tested and the supplemental herbicides (e.g., glyphosate and PSII inhibitor), and allowing further growth after such treatment, the assayed plant can be assessed for resultant injury symptoms. The extent to which the plant is allowed to grow after inhibitor treatment is in some part dependent upon the time frame of injury symptom expression.

Injury symptoms resultant from the combined herbicide treatment may be measured by several methods commonly understood in the art. For example, injury symptoms can be measured as treatment impact on: chlorosis, necrosis, growth reduction, morphological stunting, gas exchange, photosynthetic efficiency, leaf optical properties, or other stress physiology parameters commonly known in the art.

Generally, a sub-lethal rate of glyphosate will produce the visual symptom of chlorosis on most plants. If the application rate is low enough, this symptomology is transient and the plant will recover. It is thought that high rates of glyphosate create stress in Roundup Ready plants, with the level of stress being inversely related to the level of tolerance. PSII inhibitors would also cause chlorosis at sub-lethal rates. When applied in conjunction with sub-lethal application rates of glyphosate, a PSII inhibitor would accentuate chlorosis to a greater degree in plants with lower levels of glyphosate tolerance.

In one embodiment, chlorosis is typically observed from about 3 to about 5 days after treatment in plants such as corn. This injury is transient and one skilled in the art will recognize that such evaluation can be timed for maximum expression. Chlorosis can be measured in various ways. In one example, a visual estimation can be made in comparison to the untreated check. Injury can be noted as % chlorosis (0=no chlorosis, 100=complete chlorosis). One hundred percent chlorosis would correspond to the whole plant showing a complete yellowing of all tissue. As another example, chlorosis can be directly measured with a chlorophyll meter. An instrument of this type measures chlorophyll fluorescence and is therefore a more direct means of measuring chlorosis. As a further example, chlorosis can be measured by extracting chlorophyll from the leaf tissue and spectrophotometrically quantifying the amount present based upon leaf surface area or fresh weight.

Generally, a sub-lethal rate of glyphosate will produce an inhibition of growth rate in most plants. In one embodiment, growth inhibition is typically observed from about 5 to about 10 days after treatment in corn plants. In a further embodiment, growth inhibition reaches peak expression at about 8 days after treatment, and by 15 days after treatment, injury is substantially decreased. Injury measured as growth inhibition benefits from being easily quantified. Growth reduction can be noted as % growth reduction by visual estimation versus untreated plant (0=no growth reduction, 100=complete growth reduction). A more direct means is to measure the height of corn plants. Growth reduction can then be expressed as percentage of growth relative to the untreated check (height of affected plant/height of untreated check) or by simply comparing heights directly.

Other methods of characterizing the onset, progression, and severity of physiological stress symptoms associated with the application of herbicides of the present invention, such as glyphosate and PSII inhibitor will be apparent to one skilled in the art.

Various embodiments of the invention are capable of determining herbicide tolerance (e.g., glyphosate tolerance) of a plant by correlating tolerance with differential levels of injury. As shown by the provided examples, the assay methodology of the invention is capable of reproducing the historically observed relative glyphosate tolerance of the hybrid corn plants.

A correlation in biology is the extent to which two statistical variables vary together or the interdependence between two variables. See e.g. Dictionary of Biochemistry and Molecular Biology, 2d. ed. John Wiley & Sons, 1989. The determination of relationships in biological assays by means of correlation is well known to those skilled in the art.

Prior to this invention, event selection occurred in field trials by observing injury from herbicide (e.g., glyphosate), usually with treatments made later in the season, and by comparing crop yields. The drawback of such an approach was that field assays were conducted on relatively developed corn plants, thus requiring substantial time for the experiments. The method of the invention provides an assay suitable for determining herbicide tolerance, and in particular glyphosate tolerance of a plant at an earlier age than was historically possible, as well as the convenience of performing the assay in a greenhouse or growth chamber. The present invention benefits from historical methods of characterizing glyphosate tolerance in that data from these types of experiments can serve to verify the correlation described by the assay of the invention. E.g., compare Examples 1 and 2 with Example 5. Also, plants with characterized glyphosate tolerance can serve as standards against which the relative glyphosate tolerance of previously uncharacterized events may be determined. See e.g. Example 3 and 4.

Assayed plants, for example corn hybrid plants, may have known or unknown tolerance to glyphosate. Furthermore, assayed plants may be compared to plants with known or unknown glyphosate tolerance. For the purposes of the present invention, a standard plant is a plant with a characterized herbicide tolerance, and in particular glyphosate tolerance. The relative glyphosate tolerance of a standard plant can be determined by phenotypic results of event expression. Assays to characterize the phenotypic glyphosate tolerance of standard plants may take many forms including, but not limited to, analyzing changes in the chemical composition, morphology, or physiological properties of the plant. Exemplary field data characterizing the glyphosate tolerance of two Roundup Ready corn events, GA21 and NK 603, are provided in Example 5. Such techniques, and others known to those skilled in the art, can be employed to characterize the glyphosate tolerance of a plant so as to use that plant as a standard against which to determine the relative glyphosate tolerance of a plant assayed according to the methods of the invention. The same techniques can be adapted for determining a plant's tolerance to other herbicides as well.

Methods of characterizing the glyphosate tolerant phenotype of different corn event hybrids are described in, for example, commonly assigned U.S. Pat. No. 5,633,435 entitled “Glyphosate-tolerant 5-enolpyruvylshikimate-3-phosphate synthases”; U.S. Pat. No. 5,804,425 entitled “Glyphosate-tolerant 5-enolpyruvylshikimate-3-phosphate synthases”; U.S. Pat. No. 4,940,835 entitled “Glyphosate-resistant plants”; U.S. Pat. No. 5,188,642 entitled “Glyphosate-resistant plants”; and U.S. Pat. No. 6,040,497 entitled “Glyphosate resistant corn lines”; the disclosures of which are each specifically incorporated herein by reference. These characterization methods can be used in this invention as a relative scale of glyphosate tolerance of standard plants against which to compare the glyphosate tolerance of plants that have not been previously characterized.

Plants useful as standard plants of the invention include, but are not limited to, those plants genetically transformed or selected to tolerate a herbicide such as glyphosate. Plants genetically transformed or selected to tolerate glyphosate include, but are not limited to, those whose seeds are sold by Monsanto Company or under license from Monsanto Company bearing the Roundup Ready® trademark. Examples of commercially available glyphosate resistant plants useful to the invention as standard corn plants include any hybrid with the GA 21 and/or NK 603 events.

Usually, a standard plant (as the term is used herein) will be a hybrid plant with a known capacity to detoxify glyphosate and thereby resist glyphosate-induced injury. In several embodiments, the standard plant receives substantially the same treatment regime as the plant being assayed for glyphosate tolerance (see e.g. Examples 4 and 5). As used herein, treatment regime encompasses those variables which may affect the expression of injury symptoms in response to the application of glyphosate and PSII inhibitor. Examples of variables included within treatment regime include growth conditions, developmental or chronological age of plants at treatment, developmental or chronological age of plants at assessment of injury, and methods and rates of application for glyphosate and PSII inhibitor. Examples of growth conditions include relative humidity, light intensity, day length, watering schedule, nutrient supply, and planting media.

In one embodiment, the relative level of injury of an assayed plant with unknown herbicide tolerance is compared to the relative level of injury of another assayed plant of the same species with unknown herbicide tolerance. Alternatively, the assayed plant can be compared to another plant of the same species with known herbicide tolerance. In a preferred embodiment, the relative level of injury of an assayed hybrid corn plant with unknown glyphosate tolerance is compared to the relative level of injury of another assayed hybrid corn plant with unknown glyphosate tolerance. In still another preferred embodiment, the relative level of injury of an assayed hybrid corn plant with unknown glyphosate tolerance is compared to the relative level of injury of another assayed hybrid corn plant with known glyphosate tolerance (i.e., a standard corn plant).

In various embodiments, the assayed plant can be compared to one, two, three, four, or more plants with known herbicide tolerance, and in particular glyphosate tolerance. Such comparison provides a scale of tolerance. In one embodiment, the relative level of injury of an assayed hybrid corn plant is compared to the known tolerance of a corn plant hybrid known to be highly tolerant of glyphosate. As an example, the NK 603 event (as contained in, for example, the DKC 53-33 hybrid) is highly tolerant to glyphosate and typically shows no injury from glyphosate applications up to 3360 gm/ha, even applied sequentially (see e.g. Example 5).

In a further embodiment, the relative level of injury of an assayed hybrid corn plant is compared to the known tolerance of a medium glyphosate-tolerant corn hybrid. An example of a medium glyphosate-tolerant corn plant is the GA 21 event hybrid (ATCC Accession No. 209033, deposited May 14, 1997). The glyphosate tolerance phenotype of the GA 21 event is described in U.S. Pat. No. 6,040,497 entitled “Glyphosate resistant corn lines,” the disclosures of which is specifically incorporated herein by reference. Also, the glyphosate tolerance of GA 21 is characterized, for example, in Example 5.

In another embodiment, the relative level of injury of an assayed hybrid corn plant is compared to the known tolerance of a low glyphosate-tolerant corn hybrid. A low glyphosate-tolerant corn hybrid can be, for example, a corn hybrid with less glyphosate tolerance than even a GA 21 event.

In still another embodiment, the relative level of injury of an assayed hybrid corn plant is compared to a hybrid corn plant that is not RoundUp Ready (i.e., expresses only native resistance to glyphosate).

Various combinations and numbers of these high, medium, low, and non-tolerant standards are possible. In one embodiment, the assayed corn plant is compared to high and low glyphosate-tolerant corn plant hybrids. In another embodiment, the assayed corn plant is compared to high, medium, and low glyphosate-tolerant corn plant hybrids (see e.g. Example 4). In a further embodiment, the assayed corn plant is compared to high, medium, low, and non-glyphosate-tolerant corn plant hybrids. It is evident that the various iterations of possible combinations are numerous.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

The interaction of glyphosate and PSII inhibitors in Roundup Ready corn was investigated for utility of use in an assay for glyphosate tolerance. Roundup Ready corn hybrids tested were DK 580 (GA 21 event, ATCC Accession No. 209033) and DKC 53-33(NK 603 event). The NK 603 event is known to show greater tolerance to glyphosate under field conditions than the GA 21 event.

Two corn seeds were planted one inch deep per 3.5×3.5 inch plastic pot filled with commercial potting mix (Redi-earth). The potting mix was supplemented with Osmacote™ 14-14-14 slow release fertilizer at 100 gm/ft3 to optimize growth. Pots were then placed in a greenhouse (25 C day/19 C night, 14 hour day) and water was supplied through subirrigation. Plants were allowed to grow to the stage where 3 leaves were unfolded (6-9 days after planting, approximate growth stage of GS 13) prior to the application of glyphosate and photosystem II inhibitor.

Herbicide treatments consisted of application of glyphosate and PSII inhibitor. Application rates for glyphosate (Roundup UltraMAX, Monsanto, St. Louis) included: 840 gm/ha; 1680 gm/ha; and 3360 gm/ha (i.e., 0.75 lbs/A; 1.5 lbs/A; and 3.0 lbs/A). Application rates for linuron (Lorox, DuPont) included 56 gm/ha; 112 gm/ha; and 224 gm/ha. Application rates for metribuzin (Sencor, Bayer) included 56 gm/ha; 112 gm/ha; and 224 gm/ha. Treatments were broadcast applied in a research track sprayer utilizing an even flat fan spray tip. Plants were returned to the greenhouse following applications. Growth inhibition was measured 10 days after treatment (DAT). Susceptible plants showed transient chlorosis 2-4 days after treatment (DAT), which subsequently resulted in reduced growth relative to untreated plants. Growth reduction can be measured about 7-10 DAT by direct or visual estimation (0=no growth reduction, 100=complete growth reduction).

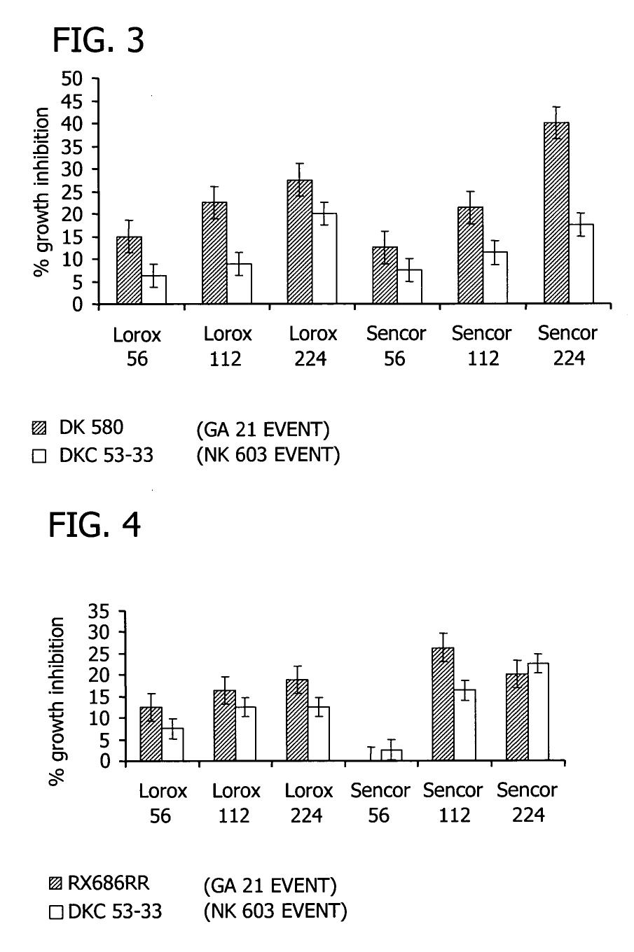

Results showed that single applications of glyphosate, metribuzin, or linuron provided minimal (<3%) to no injury in either the GA 21 event or the NK 603 event. Combinations of glyphosate with either linuron or metribuzin, however, did produce significant injury and was clearly rate-related. Injury symptomology expressed as low levels of discernable chlorosis, a minor degree of leaf necrosis (high combination rates only), and a reduction of growth. Data showed a clear rate response with both linuron (Lorox) and metribuzin (Sencor) with injury increasing as rates increased. Likewise, data showed that glyphosate injury increased as rates increased. The GA 21 event demonstrated consistently more injury in response to these combinations than the NK 603 event, suggesting that the NK 603 event has a higher degree of tolerance to glyphosate (see e.g. FIG. 3). Injury symptoms were apparent at 10 days after treatment. Injury appeared to peak at about 8 days after treatment. By 13 days after treatment, corn plants had significantly recovered.

As these results demonstrate, greater tolerance to glyphosate under field conditions was correlated with injury symptomology of combinations of glyphosate with low rates of PSII inhibitors. Thus, injury symptomology of combinations of glyphosate with low rates of PSII inhibitors in Roundup Ready corn can be used as an effective tool for selecting Roundup Ready corn events at an early stage based upon tolerance to glyphosate.

### Example 2

The interaction of glyphosate and PSII inhibitors in Roundup Ready corn was demonstrated in two corn hybrids to show the utility of use in an assay for glyphosate tolerance.

Roundup Ready corn hybrids tested were RX 686Roundup Ready (GA 21 event) and DKC 53-33(NK 603 event). Growth of plant material and treatment regime was as described in Example 1, except plants were allowed to grow to the stage where 2 leaves were unfolded (approximately GS 12) prior to the application of glyphosate and photosystem II inhibitor. Growth inhibition was measured 10 days after treatment (DAT).

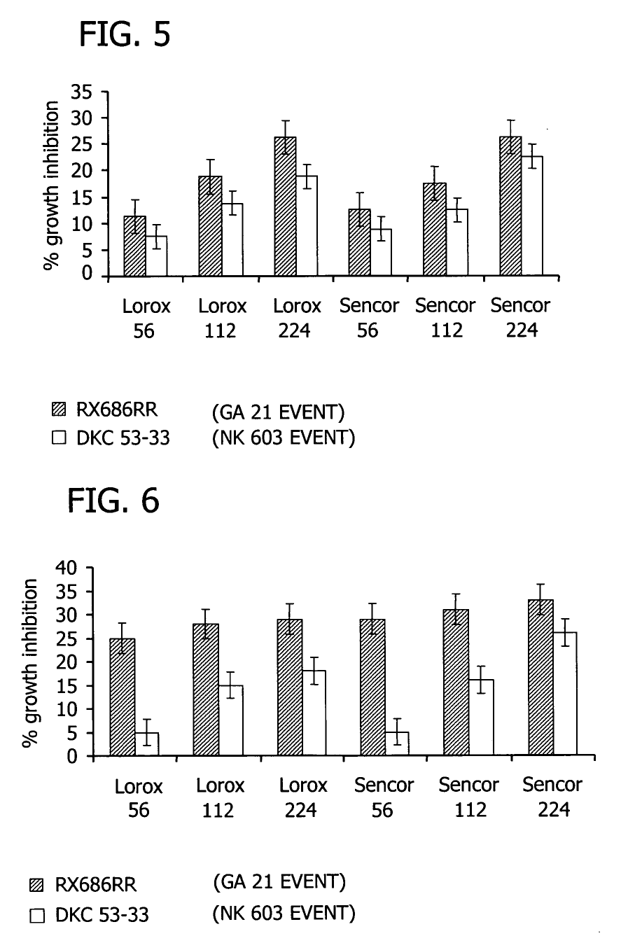

Results showed that single applications of glyphosate, metribuzin, or linuron did not produce any discernable crop injury in either of the corn hybrids. Combinations of glyphosate with either linuron or metribuzin, however, did provide significant crop injury that was rate related. Chlorosis and necrosis was observed in the combination treatments. Growth reduction data is reported in FIGS. 4-6. The two tested corn events, GA 21 (in the RX 686Roundup Ready hybrid) and NK 603 (in the DKC 53-33 hybrid), demonstrated a differential response with higher levels of injury seen in RX 686Roundup Ready. Differences between hybrids are most clearly observed at the highest application rate of glyphosate in conjunction with either linuron or metribuzin (see e.g. FIG. 6).

And so, combinations of glyphosate with low rates of PSII inhibitors induce injury in Roundup Ready corn hybrids and differences in injury between hybrids containing the NK 603 event and the GA 21 event are correlated to tolerance for glyphosate.

### Example 3

The interaction of glyphosate and PSII inhibitors in Roundup Ready corn can be demonstrated in several corn hybrids and the resulting damage compared to the damage suffered to corn plants with known levels of glyphosate tolerance (i.e., standard corn plants). In effect, this approach uses the standard corn plants to establish a standard curve of relative glyphosate resistance, where this curve can be used to assess the relative glyphosate tolerance of corn plants with unknown glyphosate tolerance.

Roundup Ready corn hybrids tested will contain glyphosate resistance events. Corn plants with the NK 603 and the GA 21 events will be selected as standard corn plants. Another event-containing hybrid that has low glyphosate tolerance will be chosen as a third standard plant. Low glyphosate tolerance for the purposes of this example constitutes a tolerance between zero tolerance and that glyphosate tolerance exhibited by the GA 21 event. The third standard plant will be characterized as to glyphosate tolerance via methods outlined in Example 5. Growth of plant material and treatment regime will be as described in Example 1, except plants will be allowed to grow to the stage where 2 leaves were unfolded (approximately GS 12) prior to the application of glyphosate and photosystem II inhibitor. At the time of application, plants of equal size will be selected for each hybrid or inbred. Growth inhibition will be measured 10 days after treatment (DAT).

Typically, results from experiments outlined above have shown a range of degree of injury from glyphosate only applications, from essentially no injury in NK 603 and GA 21 to moderate or severe injury in the third selected hybrid with the low-tolerance event. The most apparent separation among the various corn events was seen from combinations of glyphosate with the linuron rate of 112 gm/ha. The combination of glyphosate plus linuron has been observed to cause a gradient of visible injury, with the least injury to the NK 603 event, moderate injury to the GA 21 event, and severe injury to the third standard plant, selected for its known-low-tolerance to glyphosate.

Thus, selection of three standard plants can be performed such that the joint application of glyphosate and linuron produce a gradient of damage that can serve as a relative scale of reference for glyphosate tolerance of assayed plants with unknown glyphosate tolerance.

### Example 4

This example describes how to conduct a comparison of various corn events for their tolerance to glyphosate relative to the events NK 603, GA 21, and a third standard plant selected for its know-low-tolerance to glyphosate (see Example 3).

Various corn hybrids containing a glyphosate-resistance event will be selected for assay of glyphosate tolerance. Growth of plant material and treatment regime will be as described in Example 1, except: plants will be grown to the stage where only one leaf is unfolded (approximately GS 11) prior to the application of glyphosate and photosystem II inhibitor; application rates for glyphosate (Roundup UltraMAX, Monsanto, St. Louis) will be 1680 gm/ha and 3360 gm/ha; application rate for linuron (Lorox) will be 112 gm/ha; and growth inhibition will be measured 6 days after treatment (DAT).

Results for the standard events NK 603 and GA 21 are expected to show similar relative levels of glyphosate tolerance as described in the above examples (see Examples 1-2). Results for the third standard event are expected to show results consistent with its known low-tolerance for glyphosate. Consistent with observed damage effects described above, the tested events should exhibit levels of injury as a result of the combined application of glyphosate and linuron. This level of injury will be correlated to glyphosate tolerance, allowing direct comparison of the tested-events. Further, the level of injury of the tested-events will be compared to the injury levels of the standard plants of the assay. The glyphosate tolerance of the tested-events will be determined by correlation to the damage/tolerance relationship demonstrated by the standard plants. This comparison will provide an assessment of the relative glyphosate tolerance of the tested-events along a gradient of glyphosate tolerance represented by the standard plants. Based upon these data, the Roundup Ready corn events can be grouped in the following manner regarding glyphosate tolerance: Most tolerant—those similar to NK 603; Intermediate tolerant—those similar to GA 21; Least tolerant—those similar to the third standard plant selected for low-tolerance.

Therefore, differential injury from the combination of glyphosate and PSII inhibitor can be used a determinant for the glyphosate resistance of corn plant events.

### Example 5

Event selection has historically been made in field trials by observing injury from glyphosate, usually with treatments made later in the season, and by comparing crop yields. These types of experiments, and the resulting data, are useful to verify the correlation described by the glyphosate/PSII inhibitor assay of the invention.

The glyphosate tolerance of RoundUp Ready corn events NK 603 and GA 21 were characterized in field trials. Corn was planted in 36 inch rows in plots of 4 rows by 30 ft with the two center rows being harvested. The study comprised 22 locations with 4 replicates of each treatment per location and the data were pooled across locations. Corn plants were treated with glyphosate at two consecutive developmental ages of V4 and V8. Glyphosate application rates were 0.75, 1.5, and 2.23 pounds per acre (lbs/A). Application volume was 10 gallons/acre. At 10 days after treatment (DAT), the percentage of plants exhibiting chlorosis (% Chlorosis), malformed leaves (% Malform), and growth reduction (% G.R.) were determined. At 30 DAT, the percentage of plants exhibiting growth reduction was again determined. Data was expressed as the number of locations exhibiting injury greater than 9% followed by the percentage range of observed injury. Data for chlorosis, malformed leaves, and growth reduction was collected at 22 locations. Also, at harvest of mature corn plants, the yield percentage and grain moisture percentage were assessed. Data for yield and grain moisture was collected at harvest at 19 locations and expressed as the mean percentage yield or moisture with respect to controls.

Exemplary results showed that neither the NK 603 or the GA 21 corn events exhibited elevated chlorosis at 10 DAT at 0.75 lbs/A (see e.g. Table 5). But at 1.5 lbs/A, the GA 21 event exhibited elevated chlorosis while NK 603 did not. A similar data trend was observed for growth reduction at both 10 DAT and 30 DAT. Both GA 21 and NK 603 had significantly reduced yield percentages, however, the NK 603 event was less affected (see e.g. Table 6). Taken together, this data shows that while both tested events exhibit glyphosate tolerance, NK 603 is relatively more tolerant of glyphosate as compared to the GA 21 event.

This data is useful for providing a relative standard of glyphosate resistance against which to correlate the level of damage observed in connection with the assay methodology described herein.

TABLE 5
RoundUp Ready Corn Trials

| | | | | | % Growth | % Growth |
|---|---|---|---|---|---|---|
| | Glyphosate | Treatment | % Chlorosis | % Malform | Reduction | Reduction |
| Event | rate | Stage | 10 DAT | 10 DAT | 10 DAT | 30 DAT |
| GA | 0.75/0.75 | V4/V8 | None | None | None | None |
| 21 | | | | | | |
| GA | 1.5/1.5 | V4/V8 | 3(10-14) | 5(11-16) | 5(10-14) | 1(11) |
| 21 | | | | | | |
| GA | 2.25/2.25 | V4/V8 | 3(11-21) | 6(10-33) | 7(11-30) | 4(10-13) |
| 21 | | | | | | |
| NK | 0.75/0.75 | V4/V8 | None | None | None | None |
| 603 | | | | | | |
| NK | 1.5/1.5 | V4/V8 | None | 3(10-11) | None | None |
| 603 | | | | | | |
| NK | 2.25/2.25 | V4/V8 | 3(11-19) | 6(11-19) | 3(10-19) | 2(11-15) |
| 603 | | | | | | |

TABLE 6
RoundUp Ready Corn Trials

| | Glyphosate | Treatment | | % Grain |
|---|---|---|---|---|
| Event | rate | Stage | % Yield | Moisture |
| GA 21 | 0.75/0.75 | V4/V8 | 101.8 | 100.7 |
| GA 21 | 1.5/1.5 | V4/V8 | 97.8 | 101.0 |
| GA 21 | 2.25/2.25 | V4/V8 | 90.8 | 101.6 |
| NK 603 | 0.75/0.75 | V4/V8 | 103.3 | 100.2 |
| NK 603 | 1.5/1.5 | V4/V8 | 99.4 | 101.7 |
| NK 603 | 2.25/2.25 | V4/V8 | 96.6 | 103.1 |

### Example 6

The interaction of glyphosate and PSII inhibitors in Roundup Ready cotton can be demonstrated in several cotton hybrids and the resulting damage compared to the damage suffered to cotton plants with known levels of glyphosate tolerance (i.e., standard cotton plants). In effect, this approach uses the standard cotton plants to establish a standard curve of relative glyphosate resistance, where this curve can be used to assess the relative glyphosate tolerance of cotton plants with unknown glyphosate tolerance.

Cotton hybrids that are tested will contain glyphosate resistance events. Cotton plants with the 1445 or 88913 events will be selected as standard cotton plants. Another event-containing hybrid that has low glyphosate tolerance will be chosen as a third standard plant. Low glyphosate tolerance for the purposes of this example constitutes a tolerance between zero tolerance and that glyphosate tolerance exhibited by the above events. The third standard plant will be characterized as to glyphosate tolerance via methods similar to the ones outlined in Example 5 for corn. Growth of plant material and treatment regime will be as described in Example 1, except plants will be allowed to grow to the stage where 4 leaves were unfolded (approximately GS 14) prior to the application of glyphosate and photosystem II inhibitor. At the time of application, plants of equal size will be selected for each hybrid or inbred. Growth inhibition will be measured 10 days after treatment (DAT).

It is expected that the results from experiments outlined above will show a range of degree of injury from glyphosate only applications, from essentially no injury in 1445 or 88913 events to moderate or severe injury in the third selected hybrid with the low-tolerance event.

Thus, selection of three standard plants can be performed such that the joint application of glyphosate and PSII inhibitor produce a gradient of damage that can serve as a relative scale of reference for glyphosate tolerance of assayed plants with unknown glyphosate tolerance. The glyphosate tolerance is determined by assaying for, e.g., the percentage of plants exhibiting chlorosis, malformed leaves, and growth reduction.

### Example 7

The interaction of glyphosate and PSII inhibitors in Roundup Ready soybean can be demonstrated in several soybean hybrids and the resulting damage compared to the damage suffered to soybean plants with known levels of glyphosate tolerance (i.e., standard soybean plants). In effect, this approach uses the standard soybean plants to establish a standard curve of relative glyphosate resistance, where this curve can be used to assess the relative glyphosate tolerance of soybean plants with unknown glyphosate tolerance.

Soybean hybrids that are tested will contain glyphosate resistance events. Soybean plants with the GM A19788 event will be selected as standard soybean plants. Another event-containing hybrid that has low glyphosate tolerance will be chosen as a third standard plant. Low glyphosate tolerance for the purposes of this example constitute a tolerance between zero tolerance and that glyphosate tolerance exhibited by the GM A1 9788 event. The third standard plant will be characterized as to glyphosate tolerance via methods similar to the ones outlined in Example 5 for corn. Growth of plant material and treatment regime will be as described in Example 1, except plants will be allowed to grow to the stage where 4 leaves were unfolded (approximately GS 14) prior to the application of glyphosate and photosystem II inhibitor. At the time of application, plants of equal size will be selected for each hybrid or inbred. Growth inhibition will be measured 10 days after treatment (DAT).

It is expected that the results from experiments outlined above will show a range of degree of injury from glyphosate only applications, from essentially no injury in GM A19788 event to moderate or severe injury in the third selected hybrid with the low-tolerance event.

Thus, selection of three standard plants can be performed such that the joint application of glyphosate and PSII inhibitor produce a gradient of damage that can serve as a relative scale of reference for glyphosate tolerance of assayed plants with unknown glyphosate tolerance. The glyphosate tolerance is determined by assaying for, e.g., the percentage of plants exhibiting chlorosis, malformed leaves, and growth reduction.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that the several objects of the invention are achieved and other advantageous results attained. As various changes could be made in the above methods without departing from the scope of the invention, it is intended that all matter contained in the above description and shown in any accompanying figures shall be interpreted as illustrative and not in a limiting sense.

What is claimed is:

1. A method of assaying herbicide tolerance in a plant comprising:
growing the plant until a predetermined developmental age or for a predetermined interval of time;
applying a herbicide for which tolerance is being tested to the plant;
applying at least one supplemental herbicide for which tolerance is not being tested to the plant;
determining extent of injury to the plant; and
correlating the extent of injury to the plant's tolerance for the tested herbicide.

2. The method of claim 1 wherein the tested herbicide is glyphosate.

3. The method of claim 1 wherein the supplemental herbicide is a Photosystem II (PSII) inhibitor.

4. The method of claim 3 wherein the PSII inhibitor is selected from the group consisting of substituted urea, triazine, uracil, phenyl-carbamate, pyridazinone, benzothiadiazole, nitrile, and phenyl-pyridazine.

5. The method of claim 3 wherein the PSII inhibitor is selected from the group consisting of linuron, diuron, metobromuron, fluometuron, tebuthiuron, and monolinuron.

6. The method of claim 5 wherein the substituted urea PSII inhibitor is linuron.

7. The method of claim 3 wherein the PSII inhibitor is selected from the group consisting of metribuzin, atrazine, cyanazine, hexazinone, prometryne, and simazine.

8. The method of claim 7 wherein the triazine PSII inhibitor is metribuzin.

9. The method of claim 1 wherein the plant is a monocot.

10. The method of claim 9 wherein the monocot plant is selected from the group consisting of corn, rice, wheat, barley, oat, rye, buckwheat, sugar cane, onion, banana, date, and pineapple.

11. The method of claim 10 wherein the monocot plant is corn.

12. The method of claim 10 wherein the monocot plant is rice.

13. The method of claim 10 wherein the monocot plant is wheat.

14. The method of claim 1 wherein the plant is a dicot.

15. The method of claim 14 wherein the dicot plant is selected from the group consisting of cotton, soybean, canola, bean, lentil, peanut, sunflower, broccoli, alfalfa, clover, carrot, strawberry, raspberry, orange, apple, cherry, plum, parsley, coriander, dill, and fennel.

16. The method of claim 15 wherein the dicot plant is selected from the group consisting of cotton, soybean, bean, lentil, peanut, alfalfa and sunflower.

17. The method of claim 1 wherein the tested herbicide and supplemental herbicide are applied in combination.

18. The method of claim 1 wherein the tested herbicide and supplemental herbicide are applied at different times.

19. The method of claim 1 further comprising the step of comparing the extent of injury to the plant with at least one standard plant with a known tolerance for the tested herbicide, wherein the standard plant receives a treatment regime substantially similar to the plant.

20. The method of claim 19 wherein there is at least two standard plants.

21. The method of claim 19 wherein there is at least three standard plants.

22. The method of claim 19 wherein there is at least four standard plants.

23. The method of claim 19 wherein at least one standard plant is a corn plant comprising a corn event independently selected from the group consisting of NK 603 and GA 21.

24. The method of claim 19 wherein at least one standard plant is a standard corn plant with a glyphosate resistance substantially similar to corn events independently selected from the group consisting of NK 603 and GA 21.

25. The method of claim 19 wherein at least one standard plant does not comprise an event that provides tolerance to glyphosate.

26. The method of claim 1 wherein the tested herbicide is applied at a herbicidally effective rate.

27. The method of claim 26 wherein the tested herbicide is applied at about 1× to about 4× field application rate.

28. The method of claim 26 wherein the tested herbicide is glyphosate and the glyphosate is applied at a concentration of about 840 grams per hectare (gm/ha) to about 3360 gm/ha.

29. The method of claim 28 wherein the tested herbicide is glyphosate and the glyphosate is applied at a concentration of about 1680 to about 2520 gm/ha.

30. The method of claim 1 wherein at least one supplemental herbicide is applied at a concentration not sufficient to significantly injure the plant when applied independently.

31. The method of claim 30 wherein the at least one supplemental herbicide is applied at about ¼× to about 1× field application rate.

32. The method of claim 1 wherein the at least one supplemental herbicide is a PSII inhibitor and the PSII inhibitor is applied at a concentration of about 56 gm/ha to about 224 gm/ha.

33. The method of claim 1 wherein the plant is grown until a predetermined developmental age before the application of the tested herbicide and the supplemental herbicide.

34. The method claim 33 wherein the plant is a corn plant grown until a developmental age of about growth stage (GS) 11 to about GS 12 before the application of the tested herbicide and the supplemental herbicide.

35. The method of claim 1 wherein the plant is grown for a predetermined interval of time before the application of the tested herbicide and the supplemental herbicide.

36. The method of claim 35 wherein the plant is a corn plant grown for about 14 days to about 21 days before the application of the tested herbicide and the supplemental herbicide.

37. The method of claim 1 wherein the plant is grown for about 2 to about 15 days after the application of the tested herbicide and the supplemental herbicide.

38. The method of claim 37 wherein the plant is a corn plant grown for about 5 to about 10 days after the application of the tested herbicide and the supplemental herbicide.

39. The method of claim 38 wherein the corn plant is grown for about 8 days after the application of the tested herbicide and the supplemental herbicide.

40. The method of claim 1 wherein the extent of injury is measured as growth inhibition.

41. The method of claim 1 wherein the extent of injury is measured as chlorosis.

42. The method of claim 1 wherein the extent of injury is measured as necrosis.
